(19) United States
(12) Patent Application Publication
(10) Pub. No.: US 2025/0082260 A1
(43) Pub. Date: Mar. 13, 2025

(54) TISSUE CHARACTERIZATION

(71) Applicant: Whoop, Inc., Boston, MA (US)

(72) Inventors: Mostafa Ghannad-Rezaie, Malden, MA (US); Behnoosh Tavakoli, Needham, MA (US); Eric Alexander Otte, Boston, MA (US)

(21) Appl. No.: 18/885,120

(22) Filed: Sep. 13, 2024

Related U.S. Application Data
(60) Provisional application No. 63/582,414, filed on Sep. 13, 2023.

(51) Int. Cl. A61B 5/00; A61B 5/11; G16H 50/30

(52) U.S. Cl. CPC A61B 5/442; A61B 5/0048; A61B 5/0064; A61B 5/11; A61B 5/4875; A61B 5/6801; A61B 5/7271; A61B 5/742; G16H 50/30; A61B 2560/045; A61B 2560/0462

(57) ABSTRACT

The thickness of a tissue layer is calculated using optical measurements from a wearable physiological monitor. In general, a model—e.g., an analytical, empirical, or statistical model—can be created that interrelates distance from a light source (directed into the skin from the surface), light intensity, and tissue thickness. With this model, a suitable light source can be directed into skin, and measurements of light intensity at various distances along the surface of the skin from the light source can be used to calculate thickness of a tissue layer in a multi-layer structure such as the dermis over layers of fat, muscle, bone, and the like. Changes in dermal thickness can be tracked over time, and used to assess a user's health or fitness. The thickness can also support an evaluation of other characteristics such as dermis elasticity, e.g., tracking over time and correlating to health, hydration, and so forth.

### RELATED APPLICATIONS

This application claims priority to U.S. Provisional Pat. App. No. 63/582,414 filed on Sep. 13, 2023, the entire content of which is hereby incorporated by reference herein.

This application is also related to U.S. Pat. App. Pub. No. 2023/0284980 filed on Mar. 10, 2023, and U.S. Pat. App. Pub. No. US 2024/0298895 filed on Mar. 11, 2024, where the entire content of each of the foregoing is hereby incorporated by reference herein.

### TECHNICAL FIELD

The present disclosure generally relates to the characterization of layers of human tissue using a wearable physiological monitor.

### BACKGROUND

Tissue characteristics can provide useful information about health, such as the amount of body fat, the level of hydration, and so forth. There remains a need to measure characteristics such as tissue layer thickness and tissue elasticity, and to track these characteristics over time as a proxy for user health.

### SUMMARY

The thickness of a tissue layer is calculated using optical measurements from a wearable physiological monitor. In general, a model, such as an analytical, empirical, or statistical model, can be created that interrelates distance from a light source (directed into the skin from the surface), light intensity, and tissue thickness. With this model, a suitable light source can be directed into the skin, and measurements of light intensity at various distances along the surface of the skin from the light source can be used to calculate the thickness of a tissue layer in a multi-layer structure such as the dermis over layers of fat, muscle, bone, and the like. Changes in dermal thickness can advantageously be tracked over time, and used to assess the health or fitness of a user. The thickness can also be used to support an evaluation of other characteristics such as dermis elasticity, which can likewise be tracked over time and correlated to user health, hydration, and so forth.

In an aspect, a computer program product for characterizing dermis thickness disclosed herein may include non-transitory computer executable code embodied in a non-transitory computer readable medium that, when executing on one or more computing devices, causes the one or more computing devices to perform the steps of: storing a model that associates a ratio of at least two light intensity measurements with a thickness of a dermal layer in a multi-layer human tissue, where—each of the at least two light intensity measurements is obtained at one or more predetermined wavelengths, each of the at least two light intensity measurements is obtained from a surface of the multi-layer human tissue, and at least two of the at least two light intensity measurements are obtained at different predetermined distances along the surface of the multi-layer human tissue from a light source directed into the dermal layer; with a wearable physiological monitor, emitting light at the one or more predetermined wavelengths toward the skin of a user; measuring a first intensity of light at the one or more predetermined wavelengths with a first sensor; measuring a second intensity of light at the one or more predetermined wavelengths with a second sensor; and determining a dermal thickness of a dermal layer of the user by applying the first intensity and the second intensity to the model. Other aspects include corresponding computer systems, apparatus, methods, and computer programs recorded on one or more computer storage devices, each configured to perform one or more of the preceding actions.

Implementations may include one or more of the following features. The computer program product may further include code that repeats the step of determining the dermal thickness a number of times to obtain a record of changes in the dermal thickness over time. The computer program product may further include code that performs the step of calculating a health score for the user based on changes in the dermal thickness over time. The model may be empirically derived based on a correlation of measurements of the at least two light intensity measurements with a measured dermal thickness for a plurality of measurements. The model may be statistically derived based on a Monte Carlo simulation of light ray propagation in human tissue. The model may be mathematically derived based on a physical model of optical properties of human tissue. The model may include a lookup table that associates three normalized light measurements with a first thickness of the dermal layer of the multi-layer human tissue and a second thickness of a fat layer of the multi-layer human tissue. The computer program product may further include code that performs the steps of: causing a wearable physiological monitor to mechanically stimulate the surface of the skin of the user; causing the wearable physiological monitor to measure a responsive motion signal; and estimating an elasticity of the dermal layer of the user based on the responsive motion signal and the dermal thickness. The computer program product may further include code that repeats the step of estimating the elasticity of the dermal layer a number of times to obtain a record of changes in the elasticity of the dermal layer over time. The computer program product may further include code that performs the step of calculating a health score for the user based on the changes in the elasticity of the dermal layer over time. The computer program product may further include code that performs the step of estimating a hydration of the user based on the elasticity of the dermal layer. The model may be deployed on the wearable physiological monitor. The model may be deployed on a personal computing device associated with the user of the wearable physiological monitor. The model may be deployed on a server in communication with and remote from the wearable physiological monitor.

In an aspect, a method disclosed herein may include: providing a model that associates at least two light intensity measurements with a thickness of a layer in a multi-layer tissue, where the at least two light intensity measurements are (a) at a predetermined range of wavelengths and (b) at two or more predetermined distances along a surface of the multi-layer tissue; directing illumination within the predetermined range of wavelengths into a skin of a user; obtaining a plurality of light measurements by measuring an intensity of light within the predetermined range of wavelengths at the two or more predetermined distances along the surface of the skin of the user; and calculating layer thickness of a layer of a tissue of the user by applying the plurality of light measurements to the model. Other aspects include corresponding computer systems, apparatus, and computer programs recorded on one or more computer storage devices, each configured to perform one or more of the preceding actions.

Implementations may include one or more of the following features. Directing illumination may include directing illumination with a light source, and obtaining the plurality of light measurements may include obtaining the plurality of light measurements with two or more sensors positioned at the two or more predetermined distances from the light source along the surface of the skin of the user. Directing illumination may include directing illumination with two or more light sources, and obtaining the plurality of light measurements may include obtaining the plurality of light measurements with a sensor at the two or more predetermined distances from the two or more light sources along the surface of the skin of the user. The model may include a lookup table. The method may further include repeating the step of calculating the thickness of the layer a number of times to obtain a record of changes in the thickness over time. The model may be empirically derived based on a correlation of measurements of the at least two light intensity measurements with a measured dermal thickness for a plurality of dermal thicknesses. The model may be statistically derived based on a Monte Carlo simulation of light ray propagation in human tissue. The model may be mathematically derived based on a physical model of optical properties of human tissue. The layer may be a dermal layer of the skin. The layer may be a fat layer of the skin.

In an aspect, a system disclosed herein may include: a memory storing a model that associates at least two light intensity measurements with a thickness of a layer in a multi-layer tissue, where the at least two light intensity measurements are (a) at a predetermined range of wavelengths and (b) at two or more predetermined distances along a surface of the multi-layer tissue; and a wearable monitor. The wearable monitor may include: one or more light sources configured to emit light at the predetermined range of wavelengths; one or more optical sensors configured to detect light at the predetermined range of wavelengths, where the one or more light sources are positioned at the two or more predetermined distances along the surface from the one or more optical sensors when the wearable monitor is placed for use on a skin of a user; and a controller configured to direct illumination from the one or more light sources into the skin, and to obtain a plurality of light measurements from the one or more optical sensors. The system may also include a processor configured by computer executable code stored in a non-transitory computer readable medium to perform the step of calculating thickness of a tissue layer in tissue of the user by applying the plurality of light measurements to the model. The system may also include a display configured to display information related to the thickness of the layer in the skin of the user. The display may include at least one of a display on the wearable monitor, a display on a smart watch, a display on a smart phone, a display on a user device, and a display presented on a web page.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a system for tissue characterization.

FIG. 7 is a flow chart of a method for tissue characterization.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended or stated purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the disclosed embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night), etc. More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example increasing the sampling rate when signal variability increases, or during periods of relatively higher motion, or based on user input.

At the same time, continuous monitoring is not intended to exclude ordinary data acquisition interruptions such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data such as optical intensity signals, or processed data therefrom such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component or module of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as, DRAM, SRAM, EDO RAM), and so forth. Although not depicted, any of the devices or components described herein may include a computer-readable medium or other memory for storing program instructions, data, and the like.

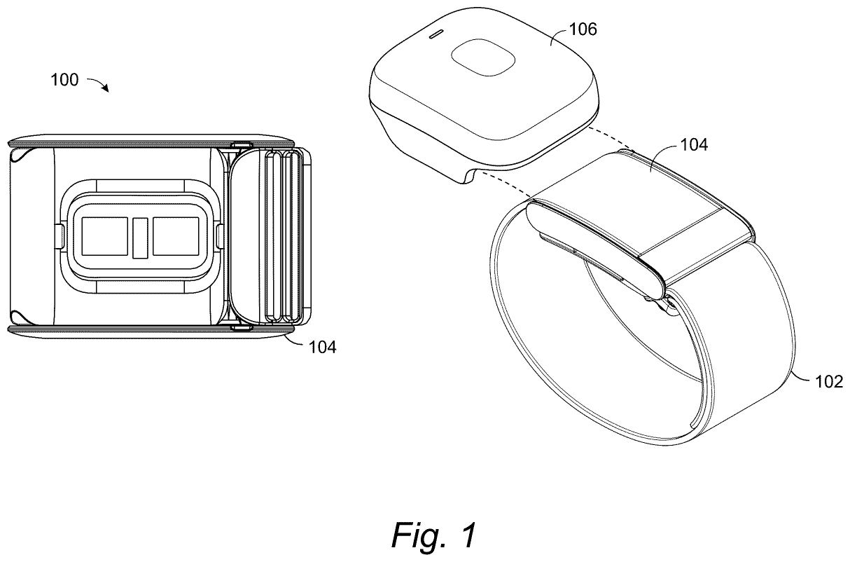

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiogram any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure (via physical pressure measurements or other means), sound, electrocardiograms, and the like, as well environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting addition functions of the wearable monitor 104. For example, the system 100 may include communications systems to support, e.g., near field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a GeoPositioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

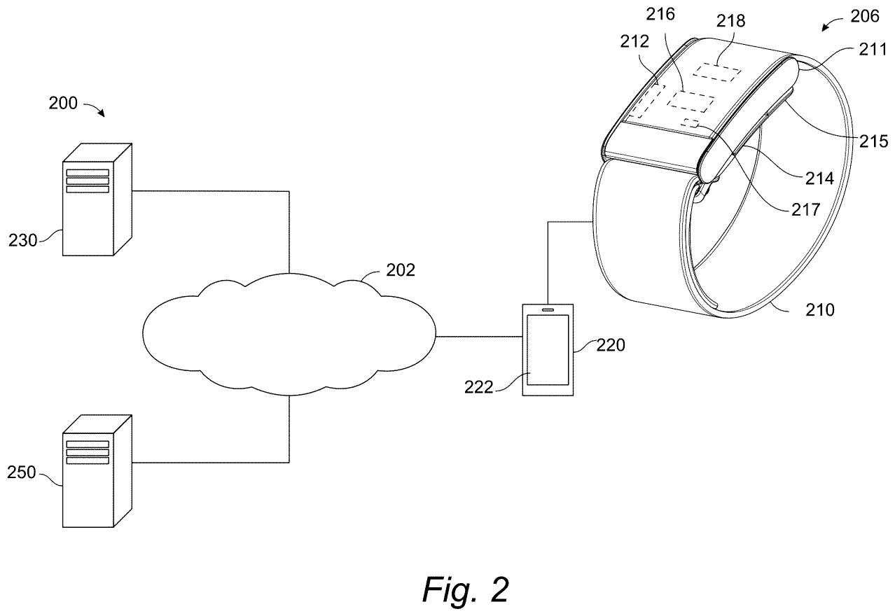

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communicating with local resources. By way of non-limiting examples, short range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicated detected motion, and/or beacons or the like to self-identify type, weight, and so forth, in order to facilitate automated detection and tracking of exercise activity with other connected devices.

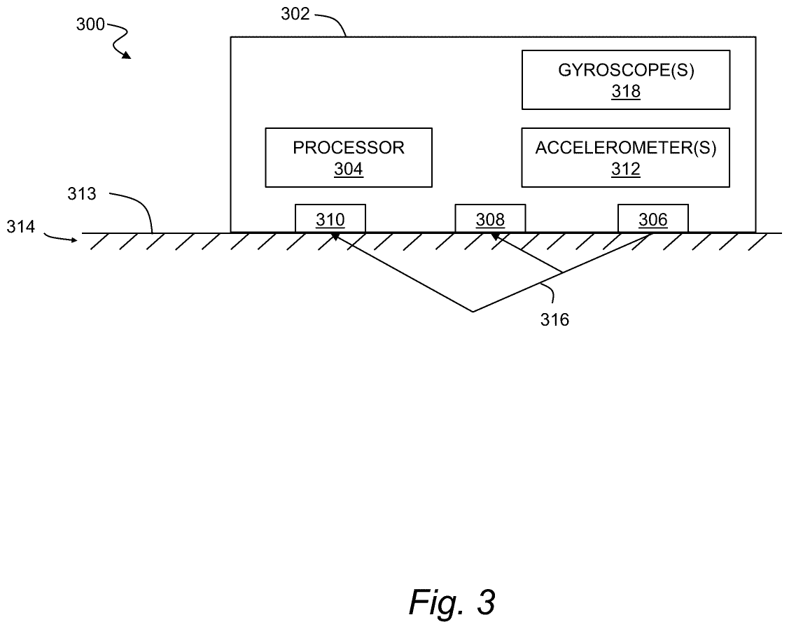

FIG. 3 shows a sensing system for a wearable monitor. In general, the system 300 may include a physiological monitor 302 with a processor 304, a light source 306, a first sensor 308 (such as a first photodetector), a second sensor 310 (such as a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, or the like described herein.

The processor 304 may be any microprocessor, microcontroller, application specific integrated circuit, or other controller or processing circuitry or combination of the foregoing suitable for controlling operation of the physiological monitor and acquiring physiological data.

The light source 306 may include one or more light emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316, where the intensity can be measured. In one aspect, the light source 306 may include light emitting diodes that emit light in the infrared or near infrared wavelength ranges, which provides good light transmission through human skin, facilitating low-power transmission of measurable illumination to the sensors 308, 310, although other illumination sources and wavelengths may also or instead be used. Other colors such as green or red light sources, such as light emitting diodes, may also or instead be used depending on the skin depth, sensed parameters of interest, and so forth.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include any optical sensors such as photodiodes, photodetectors, or any other sensor(s) responsive to illumination from the light source 306. For example, where the lights source 306 provides illumination at one or more predetermined wavelengths or range(s) of wavelengths, the sensor(s) 308 may be responsive to the wavelength, wavelengths, range of wavelengths, or ranges of wavelengths of interest for a particular measurement context. In general, the sensors 308 may include optical sensors such as broadband optical sensors, narrowband optical sensors, filtered sensors, or the like. The sensor(s) 308 may be arranged at different distances from the light source(s) 306, and or positioned over different regions on a user's skin. For example, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 310 from the light source 306. Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth.

It will also be understood that, where intensities at different distances are of interest, the system 300 may include a single light source 306 and multiple sensors 308, or multiple light sources 306 and a single sensor 308, or multiple light sources 306 and multiple sensors 308. Furthermore, depending on the purpose of the measurements, the system 300 may include three or more light sources 306, three or more sensors 308, or some combination of these. For example, where the system 300 is used to independently measure the thicknesses of two or more layers of a multi-layer tissue (e.g., a fat layer, a dermal layer, and so forth), three or more distances may be used, and may be achieved with any of a variety of combinations of sensors 308 and light sources 306.

In one aspect, the sensors 308, 310 may be linearly arranged in a straight line away from the light source 306. While this provides consistency in comparative measurements, it is not strictly required, and the sensors 308 may be displaced in any directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may obtain light measurements, e.g., by acquiring raw intensity data from the sensors 308, 310 during illumination, and perform local calculations such as pre-processing raw data for heart rate measurements, or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, and degree of musculoskeletal activation, e.g., as described herein. Other motion and orientation sensing hardware-such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

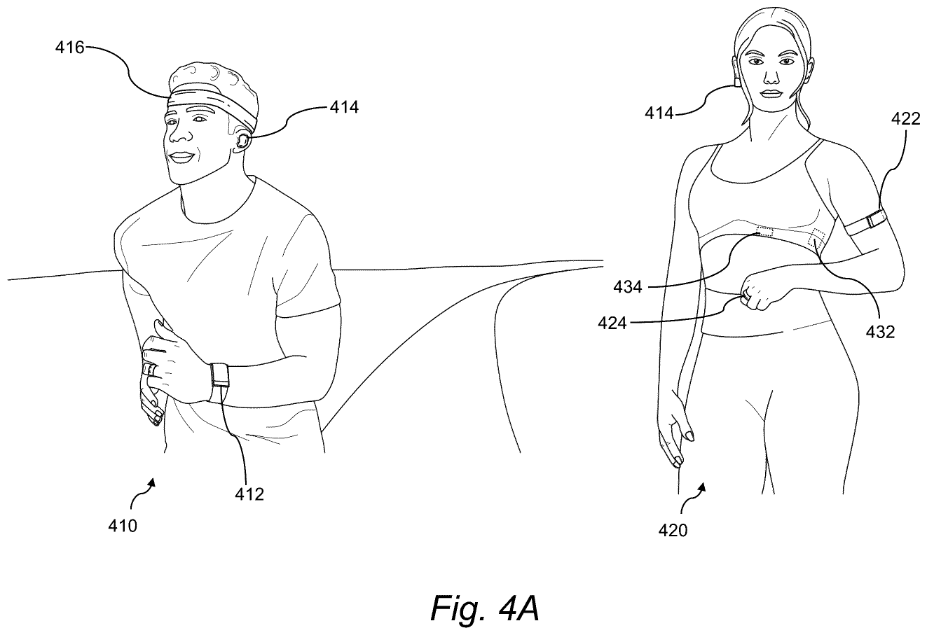

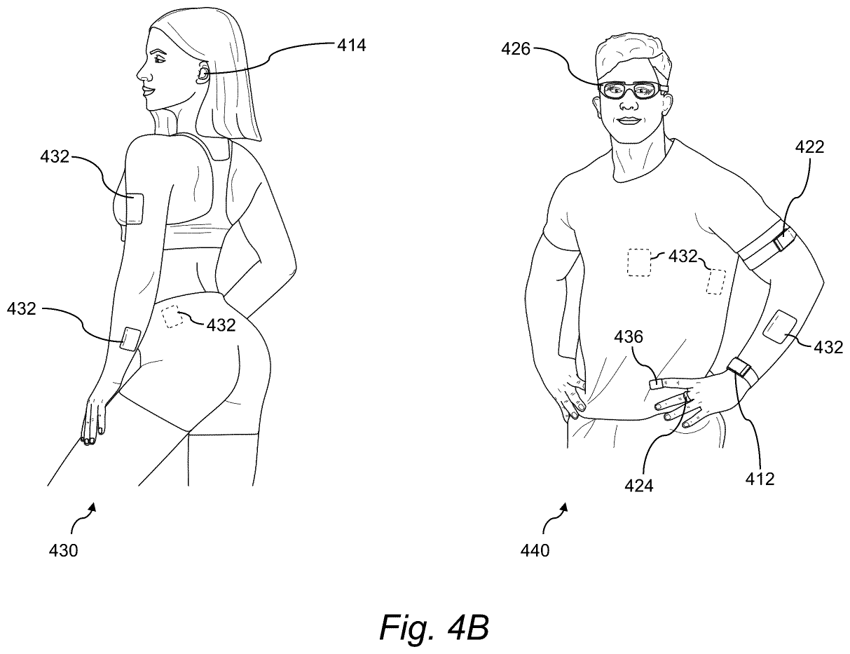

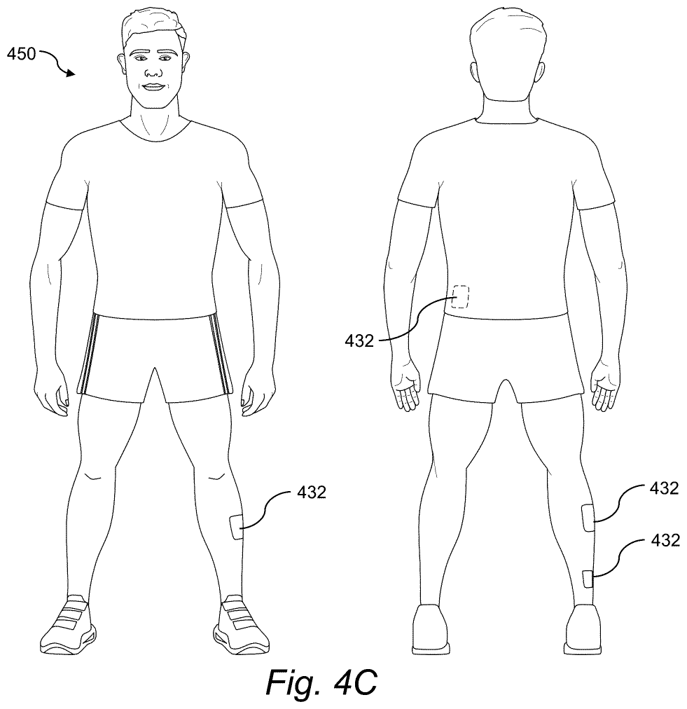

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors such as a wrist-worn device 412 (such as any described herein), an ear-worn device 414 (including on-ear devices retained with a clamp, clip, or other mechanism, and/or in-ear devices such as earbuds or the like that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large scale motion such as geographical movement and elevation changes that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors, and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to a pulse oximetry or SpO2) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smart phone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some aspects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smart phone or other computing device, to perform cooperative monitoring. For example, various monitoring techniques, such as electrocardiography blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as wi-fi or cellular signals, and converting them into usable electrical power), ambient light harvesting, and so forth. Other techniques may also or instead be used to provide external power, such as beam steering or resonant techniques for short range or medium range radio frequency power transfers. More generally, any technique or combination of techniques for powering a device, and/or for supplementing an internal power source such as a battery, with power from ambient sources may be used to power one of the monitoring devices described herein.

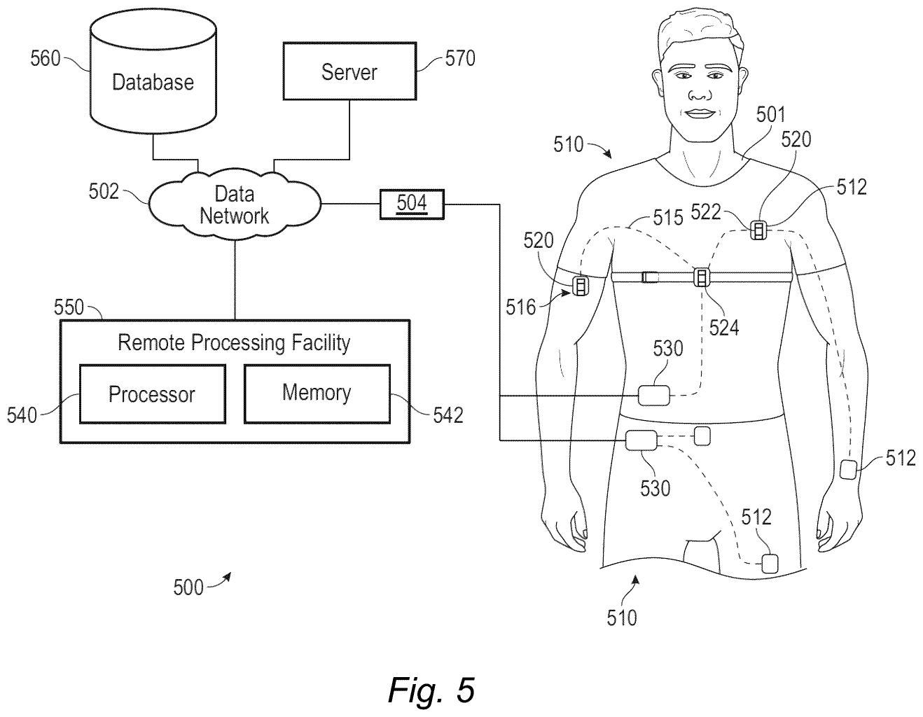

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system to be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on-capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smart phone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interactions, for example by storing data locally and transmitting the data over available local area network resources when a local access point such as a wireless access point or a relay device (such as a laptop, tablet, or smart phone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, ectroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for received light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 524 may be any as described herein, for example including any of the features of the network interface 504 described above.

The controller 530 may be configured, e.g., by computer executable code or the like, to determine a location of the module 520. This may be based on contextual measurements such as accelerometer data from the module 520, which may be analyzed by a machine learning model or the like to infer a body position. In another aspect, this may be based on other signals from the module 520. For example, signals from sensors such as photodiodes, temperature sensors, resistors, capacitors, and the like may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500 such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes, and may, for example, be used to improve the design or use of modules 520 and garments 510, either for a population, for a user type, or for a particular user.

As stated above, the system 500 may further include a processor 540 and a memory 542. In general, the memory 542 may bear computer executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like as shown in the figure. Thus, in an aspect, one or more of the processor 540 and the memory 542 is included on at least one of the plurality of modules 520. In this manner, processing may be performed on a central module, or on each module 520 independently. In another aspect, one or more of the processor 540 and the memory 542 is remote relative to each of the plurality of modules 520. For example, processing may be performed on a connected peripheral device such as smart phone, laptop, local computer, or cloud resource.

The processor 540 may be configured to assess the quality of the data received from a physiological sensor 522 of the module 520, otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources, may advantageously be used in combination to improve physiological monitoring and achieve modes of monitoring not previously available.

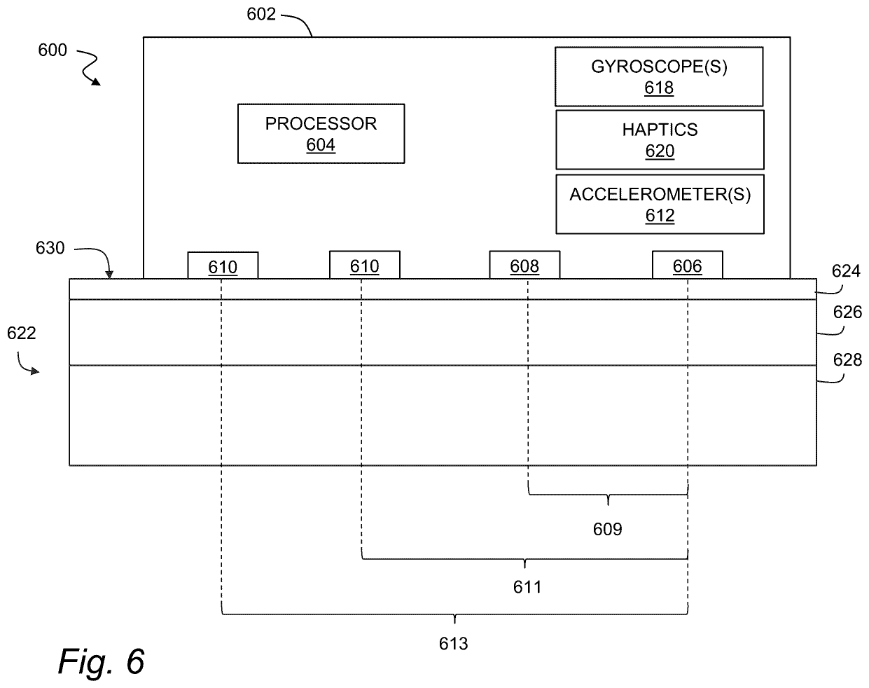

FIG. 6 shows a system for tissue characterization. In general, the system 600 may include a monitor 602 with a processor 604, an accelerometer 612, a gyroscope 618, and haptics 620, such as any described herein. The monitor 602 may be placed over a subject, e.g., over a multi-layer tissue 622 of the subject including a first layer 624 (e.g., a dermis), a second layer 626 (e.g., fat), and a third layer 628 (e.g., muscle). More generally, a classic skin model includes six layers: the stratum corneum (about 0.020 mm), the epidermis (about 0.064 mm), the melanin layer (about 0.010 mm), the basal layer (0.010 mm) (collectively, the “thin surface layers”), the dermis (about 2.000 mm), and subcutaneous fat (about 3.000 mm), and additional layers such as varying thicknesses of muscle and/or bone, depending on location. For the present discussion, this model is simplified into three layers of dermis and fat—the dominant tissue layers in terms of thickness, and tissue layers that have sufficiently homogenous mechanical and optical properties to facilitate physical modeling-along with muscle, which can be viewed as a single layer of infinite thickness for various modeling purposes. Factors such as stress, hydration, and/or blood pressure can alter the thickness and/or mechanical properties of the two top layers (dermis and fat), and changes in this thickness can provide useful information about skin health, hydration, fitness, and the like.

An optical model may be created to calculate the intensity of light measured on the surface 630 of the multi-layer tissue 622 due to a light a predetermined distance from the measurement location. For example, in one embodiment, each layer is assumed to be optically and mechanically homogenous, and that hemoglobin absorption does not affect calculations for a range of absorptions of interest. In another aspect, the effects of circulating blood can be decreased by increasing pressure on the skin to attenuate the effects of pulsation. With these constraints, the intensity of light traveling through a layer of the multi-layer tissue may be expressed as a function of wavelength, distance, and diffusion for the layer as:

$$ϕ ( ω , d , t ) = - C ( ω ) + \frac{1}{4 d π {D}_{ω , t}} {e}^{\frac{- d}{{δ}_{w , t}}}$$

$$and$$

$$D = \frac{1}{3 ( {μ}_{a} + {μ}_{s} ( 1 - g ) )}$$

$$δ = \sqrt{\frac{D}{{μ}_{a}}}$$

g=cos (θ)
θ=the scattering angle of the medium
ϕ=the intensity of light measured at a location
ω=the wavelength of the light
d=distance from the source of light to the location (along the skin surface)
t=the layer thickness for a layer of the multi-layer tissue
C(ω)=the attenuation of the light due to the thin surface layers
μₐ=the absorption coefficient for the layer
μₛ=the absorption coefficient for the layer
   From this general model, the reflected light from the multi-tissue layer structure at a point on the surface can be resolved as:

$$R ( ω , d , t ) = \frac{1}{{d}^{2}} ( \frac{1}{{δ}_{ω , t}} + \frac{1}{d} ) {e}^{\frac{- d}{{δ}_{ω , t}}}$$

In order to draw inferences about thickness, a number of measurements may be taken over a number of different distances along the surface, and then fit to this equation. Alternatively, the equation may be used to build a lookup table for a range of intensities at two or more predetermined distances. The layer thicknesses may then be retrieved based on measured intensities at a number of predetermined distances. Other techniques may also or instead be used, such as Monte Carlo simulations, regression over a number of different detectors, and so forth.

In order to obtain optical measurements to support such calculations of tissue thickness, the monitor 602 may include, e.g., a light source 606, a first optical detector 608 disposed a first distance 609 from the light source 606, a second optical detector 610 disposed a second distance 611 from the light source 606, a third optical detector 612 disposed a third distance 613 from the light source 606, and so on. Any number of different or additional source/detector pairs with different distances may be used, e.g., to improve resolution or increase the number of different layers that can be resolved. It will be understood that, while distances are generally illustrated as center-to-center (e.g., center of source to center of detector), however, any suitable configuration or standard for measuring distances may be used (e.g., closest edges, farthest edges, etc.), provided the same measurement technique is used for creating the model and applying the model.

In another aspect, the haptics 620 may be used to measure mechanical properties of the tissue, which may also be used to evaluate tissue elasticity, as described for example, in U.S. Pat. App. Pub. No. 2024/0298895, the entire content of which is hereby incorporated by reference herein. The bulk elasticity measured with, e.g., a haptic device, may then be used in combination with optically measured layer thicknesses to calculate the elasticity and/or other mechanical properties of each layer individually. As further described herein, layer thickness and elasticity may be used directly as fitness metrics, or tracked over time to detect changes in health and fitness.

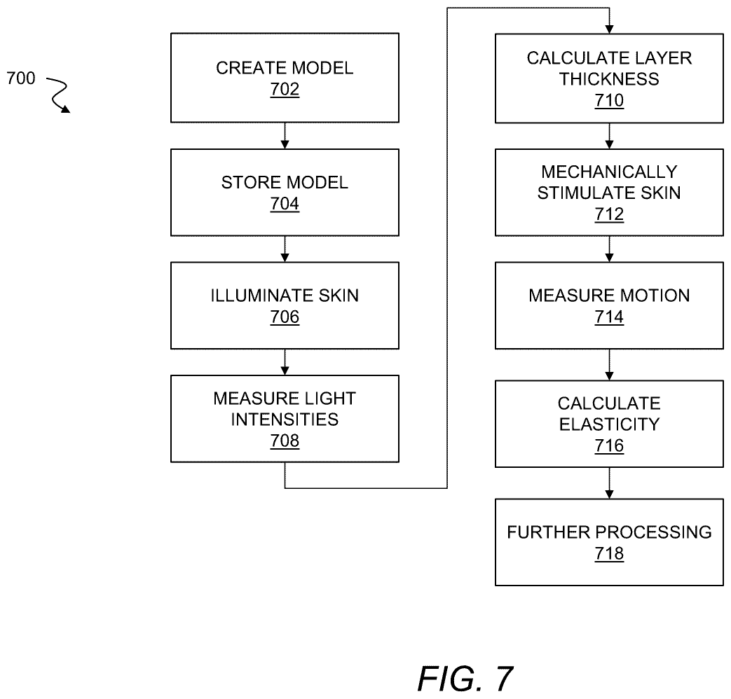

FIG. 7 is a flow chart of a method for tissue characterization. As described herein, differential light propagation through a multi-layer structure such as human tissue can be measured based on differences in a measured light intensity at different distances or different wavelengths, and can be used to characterize the thickness (and/or other properties) of one or more layers of the multi-layer structure. For example, a model such as any of the models described herein may be applied to relate measured intensities, or a ratio of two measured intensities, to a tissue property such as tissue thickness, or more specifically to the thickness of a layer of tissue in a multi-layer structure. With this model, a light source may be directed into the skin, and then two or more measurements at two or more distances (corresponding to the modeled distances) from the light source along a surface of the skin can be used to calculate or estimate the thickness of the layer of interest.

As shown in step 702, the method 700 may include creating a model to evaluate a tissue property based on optical measurements. For example, the model may include a model to calculate a thickness of a layer in a multi-layer tissue structure based on measurements of light intensity at two or more distances from a light source, such as a model that associates a ratio of at least two light intensity measurements with a thickness of a dermal layer or a fat layer in a multi-layer human tissue. A variety of models and modeling techniques may be employed to derive tissue properties such as layer thickness from differential light intensity measurements. In general, the optical properties of various surface layers such as the stratum corneum, the epidermis, the melanin layer, the basal layer, the dermis, and subcutaneous fat, do not typically vary significantly from person to person, or from location to location on the body of a particular person, so bulk optical properties may usefully be employed in modeling, and an empirical model developed with a population may usefully be applied across most users. It will be noted that, notwithstanding any consistency in optical properties, a reasonably large population is still helpful for building a model in this context because a number of different layer thicknesses may be useful for creating a robust model, particularly where the variety of possible thicknesses might not be reliably found in a single individual or small group of individuals.

In one aspect, a model may be physically and/or analytically derived, such as the model described with reference to FIG. 6. This may be used to calculate thickness by fitting a group of measurements at different distances to the equations for attenuation to derive values for a thickness of each layer of tissue. In another aspect, the analytical model may be used to populate a lookup table in order to mitigate the requirement for complex calculations at the time of a particular measurement.

In another aspect, a model may be empirically derived based on a correlation of measurements of the at least two light intensity measurements with a property of interest, such as a measured dermal thickness or fat thickness and corresponding optical measurements for a plurality of separate tissue layer thicknesses. That is, a range of actual measurements may be taken of both tissue thicknesses and light intensity at different distances, or light intensity at different distances may be measured for a range of multi-layer tissues with known individual thickness layers. In one aspect, the model may include a statistical model such as a linear regression model or the like that relates a dependent variable (e.g., layer thickness) with a number of independent variables (e.g., intensity at distance d1, distance d2, and so forth) through a model equation and a number of calculated regression coefficients. The regression model may also use exponential or power functions as necessary or helpful to capture the physical nature of tissue properties over a range of measurements. In another aspect, the acquired data may be used to create a lookup table that associates location-based intensity measurements with tissue layer thickness(es). In this case, interpolation may be used to evaluate layer thicknesses when actual light intensity measurements vary from lookup table indexes.

While one layer models are described, it will be understood that the techniques described herein may also or instead be used to create models for inferring the thickness of two or more tissue layers. For example, with light intensity measurements at three or more distances from a source, the resulting data may be used to model a two-layer tissue structure (or a three layer tissue structure where the third layer is infinitely deep), such as a structure with a dermis layer and a fat layer (with underlying muscle or bone). In one aspect, a lookup table may be created based on a collection of actual measurements that associates a combination of at least three light measurements (e.g., at different distances from a single source, or across multiple sources and sensors) with a first thickness of the dermal layer and a second thickness of a fat layer of the multi-layer human tissue. In this case, the model inputs may be individual intensity ratios (e.g., d1/d2, d2/d3, d1/d3), or some other normalized measurement or representation of intensities from the three (or more detectors).

While a lookup table or regression model may be derived from empirical data, other techniques may also or instead be employed to mathematically derive a suitable model based on, e.g., optical properties of various tissue layers. In one aspect, a model may be statistically derived based on a Monte Carlo simulation of light ray propagation in human tissue. That is, a simulation of the multi-layer tissue may be created, and the path of light rays through the simulated tissue may be calculated at various angles of incidence. By summing the number of simulated photons or light rays arriving at a sensor, and possibly accounting for angle of incidence, the theoretical intensity of received light from a light source at one or more optical sensor locations can be calculated. By using a single light source and a ratio of received light intensities, certain calibration challenges (e.g., the relative intensity of two different sources) can advantageously be avoided. In another aspect, the model may be mathematically derived, e.g., as described herein, based on a physical model of optical properties of human tissue. For example, by using known bulk optical tissue properties for different layers (e.g., absorption coefficients, scattering coefficients) along with assumptions about the optical homogeneity of each tissue layer, an expression for the surface optical intensity as a function of distance can be derived.

Other models and simplifications may also or instead be used. For example, in one aspect, a value for an expected intensity may be calculated using a simplified, single layer diffusion model for two extreme cases-first, that the entire measured layer is skin (based on known, bulk optical properties of skin), and second, that the entire measured layer is fat (based on known, bulk optical properties of fat). While neither assumption will be absolutely true, it is true that beyond a certain skin depth, substantially all of the returning, measurable light is scattered and reflected from the current layer. Thus, with these two boundary conditions, a simple mathematical model such as a linear model, exponential model, or quadratic model, may be used to monotonically associate, e.g., a ratio of distance (along the surface, from the source) and intensity with layer thickness.

As noted above, while the description herein emphasizes the measurement of a single tissue layer thickness, e.g., for dermis, the techniques may be generalized to two or more thicknesses within a multi-layer tissue structure, provided sufficient data is available, in particular measurements at a sufficient number of different distances to resolve individual layer thicknesses.

It will also be noted that the techniques described herein may be further augmented and refined. For example, different models may be created for different body locations. For example, a model for measurements with a wrist-worn physiological monitor may be different than a model for a monitor in a bicep band or an elastic waist band of shorts or underwear, particularly where the skin, fat, and muscle are expected to have significantly different thicknesses and/or mechanical properties. Thus, a number of different models may be created, e.g., using the techniques described herein, for each position where a monitor might be expected to be placed, and a suitable model may be selected by either manually or automatically identifying the location of the monitor. In another aspect, a default model may be provided for use in situations where a monitor might be at a number of different locations, but the actual location is unknown.

As shown in step 704, the method 700 may include storing the model in any suitable memory location for performing calculations as further described herein. In one aspect, the model may be stored on a wearable monitor, e.g., where the wearable monitor locally performs layer thickness calculations based on optical measurements. In another aspect, the model may be stored on a user device such as a desktop computer, laptop computer, tablet, smartphone, smart watch, or other device coupled in a communicating relationship with the wearable monitor and configured to receive optical measurements from the wearable monitor and apply the model to calculate layer thickness based on the optical measurements. The model may also or instead be stored on a remote processing resource such as a web server or the like and configured to receive the optical measurements and apply the model to calculate layer thickness. In general, each of these configurations may provide certain advantages. For example, by storing and applying the model on a remote resource, more complex models may be created and applied, and the model may be updated more frequently without requiring changes to an application executing on a local user device, or changes to firmware on the wearable monitor. Thus more generally, the model may be deployed on a wearable physiological monitor, on a personal computing device associated with the user of the wearable physiological monitor, or on a server in communication with and remote from the wearable physiological monitor, as well as any combination of these suitable for providing timely data and analysis to the user.

In one embodiment, storing the model may include storing a model that associates a ratio of at least two light intensity measurements with a thickness of a dermal layer in a multi-layer human tissue. In general, the model may usefully be matched to a particular measurement context provided, e.g., by the configuration and capabilities of a monitoring device. For example, in one aspect, the model may be matched to the measurement context by using a model and a physical monitoring configuration where: each of the at least two light intensity measurements is obtained at one or more predetermined wavelengths, each of the at least two light intensity measurements is obtained from a surface of the multi-layer tissue, and at least two of the at least two light intensity measurements are obtained at different ones of the predetermined distances along the surface of the multi-layer tissue from a light source directed into the dermal layer. More generally, the method 700 may include providing a model that associates at least two light intensity measurements with a thickness of a layer in a multi-layer human tissue or other multi-layer tissue or structure, where the at least two light intensity measurements are (a) at a predetermined range of wavelengths and (b) at two or more predetermined distances along a surface of the multi-layer tissue. In this context, providing the model may include creating the model, storing the model in a memory for use in calculating tissue properties, or some combination of these.

As shown in step 706, the method 700 may include illuminating the skin. For example, this may include, emitting light with a light source of a wearable physiological monitor at one or more predetermined wavelengths (or ranges of wavelengths) into the skin of a user, or more generally toward the surface of the multi-layer tissue of a user. This may include one or more predetermined wavelengths or ranges of wavelengths that were used to create the model described above. In one aspect, this may include illuminating the skin with one or more light emitting diodes, or any other light source or combination of light sources described herein. For example, in a single light source embodiment, directing illumination may include directing illumination with a light source, in which case intensity may be measured by obtaining the plurality of light measurements with two or more sensors positioned at the two or more predetermined distances from the light source along the surface of the skin. In another aspect where multiple light sources or wavelengths are used, directing illumination may include directing illumination with two or more light sources, in which case intensity may be measured by obtaining a plurality of light measurements with a sensor at the two or more predetermined distances from the two or more light sources along the surface of the skin. It will be understood that other techniques may also or instead be used. For example, a single broadband light source may be used, with wavelength-specific photodetectors used to measure intensity at different wavelengths (or wavelength ranges).

As shown in step 708, the method 700 may include measuring light intensities. For example, this may include measuring a first intensity of light at the one or more predetermined wavelengths with a first sensor, and measuring a second intensity of light at the one or more predetermined wavelengths with a second sensor, e.g., where each of the first sensor and the second sensor is spaced apart from the light source along a surface of the skin by two different ones of the predetermined distances used in the model described above. While two sensors and distances are described, it will be understood that three or more different intensity measurements at three or more different distances may also or instead be used, e.g., as an error check on a single layer thickness measurement or the like, or as an additional data point to permit resolution of additional layer thicknesses (or other tissue properties). Thus, more generally, the method 700 may include obtaining a plurality of light measurements by measuring an intensity of light within the predetermined range of wavelengths at two or more predetermined distances along the surface of the skin of the user for use with a model, such as any of the models described herein, to evaluate a tissue property.

As shown in step 710, the method 700 may include calculating a layer thickness or other property. This may include calculating the layer thickness for a tissue layer of a user with any of the optical techniques described herein. For example, the method 700 may include determining a dermal thickness of the multi-layer tissue of the user by measuring a first intensity of light (at a known wavelength and a first predetermined distance) and a second intensity of light (at a known wavelength and a second predetermined distance) and applying these measurements to the model. More generally, the method 700 may include calculating the thickness of the tissue layer in the skin of the user by applying a plurality of measurements of light intensity to a corresponding model, such as any of the light measurements and models described herein. Where the property of interest is a tissue layer thickness, the layer may, for example, be a dermal layer of the skin, a fat layer of the skin, or any other discrete tissue layer that can be optically sensed using the techniques described herein.

It will be understood that the model, and the corresponding calculations, may be modified in a number of ways. In one aspect, measurements may be taken at each of a number of different wavelengths. As noted herein, different sensed wavelengths may conceptually be used in place of different sensor distances to resolve tissue thickness. However, measurements at different wavelengths may also or instead be used in addition to same-wavelength measurements at different distances, such as to mitigate the effects of blood volume and oxygenation, e.g., based on different penetration depths of different wavelengths and different absorption spectra for oxygenated and deoxygenated blood. In another aspect, different wavelengths may be used in addition to different measurement distances (along with a corresponding model) as additional data to more accurately resolve a layer thickness or other tissue property of interest.

As shown in step 712, the method 700 may include mechanically stimulating the surface of the multi-layer tissue of the user. For example, this may include vibrating the wearable physiological monitor with a haptic output device of the monitor, or with any other source of mechanical vibrational energy. In one aspect, the vibration may be varied in frequency, e.g., with a CHIRP signal or the like, in order to evaluate the frequency response of the underlying tissue. In general, this technique may be used to model and measure mechanical properties of the tissue such as elasticity. Where the measurement yields a bulk mechanical property such as elasticity for a multi-layer tissue, the property may be derived for each individual layer using the optically measured layer thicknesses.

As shown in step 714, the method 700 may include measuring a responsive motion signal to the vibration from the haptic device, e.g., with an inertial measurement unit or other motion sensor or sensing device/system on the wearable monitor.

As shown in step 716, the method 700 may include calculating an elasticity of the tissue underlying the wearable monitor based on the vibration applied in step 712 and the responsive motion detected in step 714. For example the stimulus and the response may be measured over time, and used to calculate the tissue elasticity. A variety of techniques may be used to calculate elasticity—e.g., a modulus of elasticity expressed as a ratio of the stress (force/area) to the strain (change in length relative to original length)-based on a mechanical stimulus such as a haptic input and a resulting movement or deformation as measured, e.g., with an accelerometer, gyroscope, or the like. Any such techniques may be employed with the method 700 herein to determine tissue elasticity. In one aspect, the method 700 may advantageously use the measured layer thickness(es) obtained as described above to resolve an elasticity of one or more specific layers. A variety of techniques may be used to determine a layer-specific elasticity in this context. For example, this may include creating a linear system of equations based on the force and displacement for each layer, along with certain boundary conditions (e.g., a stationary bottom surface of the multi-layer structure), assumptions (e.g., homogenous mechanical properties for each tissue layer), and additional information (e.g., that the sum of forces applied to all layers equals the contact force on the surface). By solving this system of equations, a modulus of elasticity for each individual layer can be calculated. Where dermal elasticity is the property of interest, calculating the elasticity may include estimating the thickness of a dermal layer of the multi-layer tissue (and any other thicknesses of adjacent layers needed to resolve elasticity) using the optical techniques described herein, and then using the mechanical techniques described herein to calculate, e.g., a modulus of elasticity for a dermal layer of known thickness. Similarly, where elasticity of the fat layer is the property of interest, the method 700 may also or instead include determining an elasticity of the fat layer by determining the thickness of the fat layer (and any other adjacent layers needed to resolve elasticity) with optical techniques, and then calculating a modulus of elasticity for a fat layer of known thickness.

As shown in step 718, the method 700 may include one or more additional processing steps. For example, in one aspect, the method 700 may include repeating the step of determining the dermal thickness a number of times to obtain a record of changes in the dermal thickness over time. For example, this may include daily measurements of dermal thickness for a user based on optical measurements as described herein. These daily measurements may, e.g., be stored on the wearable monitor, stored on a user device, and/or stored on a remote resource such as a web server, and used to provide a longitudinal evaluation of skin thickness, potentially as a proxy for user health. For example, dermal thickness can be related to health in a variety of ways. Certain chronic conditions systemic lupus erythematosus (SLE) or systemic sclerosis (scleroderma) can cause skin changes, including alterations in dermal thickness. In scleroderma, for instance, the skin thickens due to excess collagen deposition. Other skin disorders such as atopic dermatitis can affect the dermal and epidermal thickness, and chronic inflammation can lead to skin changes, including thickening in certain areas due to scratching. On the other hand, postmenopausal women often experience thinning of the skin due to reduced estrogen levels. Other conditions like Cushing's syndrome, where there is an overproduction of cortisol, can also lead to skin thinning. Other health related factors such as environmental exposure to ultraviolet radiation, pollutants, and smoke can affect skin health and lead to premature aging or thinning, while nutritional deficiencies such as vitamin C, vitamin A and certain proteins can lead to changes in skin thickness and overall health. Where skin thickness, and more specifically changes in skin thickness over time, can be demonstrated as a proxy for health, measured changes in the skin thickness can be used as a basis for calculating a health score for the user, e.g., based on changes in the dermal thickness over time.

Skin elasticity can also serve as a proxy for health, and may be used to evaluate user health based on changes over time. Loss of elasticity is a common sign of aging, and can also be affected by various health and environmental factors such as smoking, sun exposure, nutrition, weight change, hormonal changes, hydration, and certain medical conditions (e.g., Marfan syndrome or Ehlers-Danlos syndrome). In cases where changes in skin elasticity can be demonstrated as correlated to health changes, longitudinal monitoring of elasticity can facilitate health scoring or other observations, recommendations, and the like. Thus in one aspect, the method 700 may include additional processing such as repeating the step of estimating the elasticity of the dermal layer a number of times to obtain a record of changes in the elasticity of the dermal layer over time. The method 700 may also include calculating a health score for the user based on the changes in the elasticity of the dermal layer over time. In another aspect, the method 700 may include estimating a hydration of the user based on the elasticity of the dermal layer, which may be usefully monitored by performance athletes and the like on a regular basis to ensure proper hydration. Coaching can also be provided based on hydration, e.g., by advising a user when hydration appears adequate or appropriate, and/or by recommending an increased intake of water or other liquids suitable for increasing hydration when the measured hydration appears low. In this case, coaching may also be adapted based on the time relationship between hydration factors (such as diet, exercise, medication, illness, fluid intake, etc.) and measured hydration.

Similarly, it may be useful to measure the thickness of a fat layer over time. A certain level of body fat is essential for normal physiological functions, including regulating body temperature, cushioning organs, and storing energy. However, excess fat can indicate increased risk of cardiovascular disease, type 2 diabetes, sleep apnea, fatty liver disease, and so forth. At the same time, low body fat may indicate, or lead to, hormonal imbalances, osteoporosis, weekend immune system, and so forth. In some instances, it may be possible to estimate total body fat, body mass index, or the like, from a localized fat thickness measurement. However, even where it is not possible to derive a health-related measurement from an instantaneous, location-specific measurement of the thickness of a fat layer, tracking the thickness of a fat layer over time may permit the identification of conditions approaching excess or insufficient body fat, and may correspondingly be used as a coaching metric or an indicator or signal for recommending testing, lifestyle changes, and so forth. Thus, the method 700 described herein may include periodically measuring a thickness of a fat layer, storing a longitudinal history of fat layer thickness for a user, and analyzing the history of fat layer thickness to provide coaching recommendations for the user. Similarly, fat elasticity may be an indicator of hydration, nutritional deficiencies, and the like, and may be useful as a health and wellness metric for a user, either as an instantaneous measurement, or as a directional indicator over time for health-related issues.

According to the foregoing, there is also described herein a system for tissue characterization. The system may generally include a memory, a wearable monitor, and a processor. The memory may store a model that associates at least two light intensity measurements with a thickness of a layer in a multi-layer human tissue, where the at least two light intensity measurements are (a) at a predetermined range of wavelengths and (b) at two or more predetermined distances along a surface of the multi-layer tissue. This may, for example, include any of the models described herein. The wearable monitor may include any of the physiological monitoring devices described herein. For example, the wearable monitor may include one or more light sources configured to emit light at the predetermined range of wavelengths, one or more optical sensors configured to detect light at the predetermined range of wavelengths, where the one or more light sources are positioned at the two or more predetermined distances along the surface from the one or more optical sensors when the wearable monitor is placed for use on a skin of a user, and a controller configured to direct illumination from the one or more light sources into the skin, and to obtain a plurality of light measurements from the one or more optical sensors. The processor may be the controller of the wearable monitor, or some other processor such as a processor on a user device or a remote computing resource such as a web server. In general, the processor may be configured by computer executable code stored in a non-transitory computer readable medium to perform the step of calculating the thickness of the layer in the skin of the user by applying the plurality of light measurements to the model, e.g., using any of the techniques described herein.

In one aspect, the system may further include a display configured to display information related to the thickness of the layer in the skin of the user, which may include one or more of a display on the wearable monitor, a display on a smart watch, a display on a smart phone, a display on a user device, and a display presented on a web page.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A computer program product for characterizing dermis thickness, the computer program product comprising non-transitory computer executable code embodied in a non-transitory computer readable medium that, when executing on one or more computing devices, causes the one or more computing devices to perform the steps of:
storing a model that associates a ratio of at least two light intensity measurements with a thickness of a dermal layer in a multi-layer human tissue, wherein:
each of the at least two light intensity measurements is obtained at one or more predetermined wavelengths,
each of the at least two light intensity measurements is obtained from a surface of the multi-layer human tissue, and
at least two of the at least two light intensity measurements are obtained at different predetermined distances along the surface of the multi-layer human tissue from a light source directed into the dermal layer;
with a wearable physiological monitor, emitting light at the one or more predetermined wavelengths toward the skin of a user;
measuring a first intensity of light at the one or more predetermined wavelengths with a first sensor;
measuring a second intensity of light at the one or more predetermined wavelengths with a second sensor; and
determining a dermal thickness of a dermal layer of the user by applying the first intensity and the second intensity to the model.

2. The computer program product of claim 1, further comprising code that performs the step of repeating the step of determining the dermal thickness a number of times to obtain a record of changes in the dermal thickness over time.

3. The computer program product of claim 2, further comprising code that performs the step of calculating a health score for the user based on changes in the dermal thickness over time.

4. The computer program product of claim 1, wherein the model is empirically derived based on a correlation of measurements of the at least two light intensity measurements with a measured dermal thickness for a plurality of measurements.

5. The computer program product of claim 1, wherein the model is statistically derived based on a Monte Carlo simulation of light ray propagation in human tissue.

6. The computer program product of claim 1, wherein the model is mathematically derived based on a physical model of optical properties of human tissue.

7. The computer program product of claim 1, wherein the model includes a lookup table that associates three normalized light measurements with a first thickness of the dermal layer of the multi-layer human tissue and a second thickness of a fat layer of the multi-layer human tissue.

8. The computer program product of claim 1, further comprising code that performs the steps of:
causing a wearable physiological monitor to mechanically stimulate the surface of the skin of the user;
causing the wearable physiological monitor to measure a responsive motion signal; and
estimating an elasticity of the dermal layer of the user based on the responsive motion signal and the dermal thickness.

9. The computer program product of claim 8, further comprising code that performs the step of repeating the step of estimating the elasticity of the dermal layer a number of times to obtain a record of changes in the elasticity of the dermal layer over time.

10. The computer program product of claim 9, further comprising code that performs the step of calculating a health score for the user based on the changes in the elasticity of the dermal layer over time.

11. The computer program product of claim 8, further comprising code that performs the step of estimating a hydration of the user based on the elasticity of the dermal layer.

12. The computer program product of claim 1, wherein the model is deployed on the wearable physiological monitor.

13. The computer program product of claim 1, wherein the model is deployed on a personal computing device associated with the user of the wearable physiological monitor.

14. The computer program product of claim 1, wherein the model is deployed on a server in communication with and remote from the wearable physiological monitor.

15. A method comprising:
providing a model that associates at least two light intensity measurements with a thickness of a layer in a multi-layer tissue, wherein the at least two light intensity measurements are (a) at a predetermined range of wavelengths and (b) at two or more predetermined distances along a surface of the multi-layer tissue;
directing illumination within the predetermined range of wavelengths into a skin of a user;
obtaining a plurality of light measurements by measuring an intensity of light within the predetermined range of wavelengths at the two or more predetermined distances along the surface of the skin of the user; and
calculating layer thickness of a layer of a tissue of the user by applying the plurality of light measurements to the model.

16. The method of claim 15, wherein directing illumination includes directing illumination with a light source, and wherein obtaining the plurality of light measurements includes obtaining the plurality of light measurements with two or more sensors positioned at the two or more predetermined distances from the light source along the surface of the skin of the user.

17. The method of claim 15, wherein directing illumination includes directing illumination with two or more light sources, and wherein obtaining the plurality of light measurements includes obtaining the plurality of light measurements with a sensor at the two or more predetermined distances from the two or more light sources along the surface of the skin of the user.

18. The method of claim 15, wherein the model includes a lookup table.

19. The method of claim 15, further comprising repeating the step of calculating the thickness of the layer a number of times to obtain a record of changes in the thickness over time.

20. The method of claim 15, wherein the model is empirically derived based on a correlation of measurements of the at least two light intensity measurements with a measured dermal thickness for a plurality of dermal thicknesses.

21. The method of claim 15, wherein the model is statistically derived based on a Monte Carlo simulation of light ray propagation in human tissue.

22. The method of claim 15, wherein the model is mathematically derived based on a physical model of optical properties of human tissue.

23. The method of claim 15, wherein the layer is a dermal layer of the skin.

24. The method of claim 15, wherein the layer is a fat layer of the skin.

25. A system comprising:
a memory storing a model that associates at least two light intensity measurements with a thickness of a layer in a multi-layer tissue, wherein the at least two light intensity measurements are (a) at a predetermined range of wavelengths and (b) at two or more predetermined distances along a surface of the multi-layer tissue;
a wearable monitor including:
one or more light sources configured to emit light at the predetermined range of wavelengths,
one or more optical sensors configured to detect light at the predetermined range of wavelengths, wherein the one or more light sources are positioned at the two or more predetermined distances along the surface from the one or more optical sensors when the wearable monitor is placed for use on a skin of a user, and
a controller configured to direct illumination from the one or more light sources into the skin, and to obtain a plurality of light measurements from the one or more optical sensors; and
a processor configured by computer executable code stored in a non-transitory computer readable medium to perform the step of calculating thickness of a tissue layer in tissue of the user by applying the plurality of light measurements to the model.

26. The system of claim 25, further comprising a display configured to display information related to the thickness of the layer in the skin of the user.

27. The system of claim 26, wherein the display includes at least one of a display on the wearable monitor, a display on a smart watch, a display on a smart phone, a display on a user device, and a display presented on a web page.
